(19) United States
(12) Patent Application Publication
(10) Pub. No.: US 2026/0094727 A1
(43) Pub. Date: Apr. 2, 2026

(54) PATH ESTIMATION DEVICE, PATH ESTIMATION METHOD, AND RECORDING MEDIUM

(71) Applicant: NEC Corporation, Tokyo (JP)

(72) Inventors: Yuki KOSAKA, Tokyo (JP); Kentaro NAKAHARA, Tokyo (JP); Fumiyuki NIHEY, Tokyo (JP); Kosuke NISHIHARA, Tokyo (JP); Mana HASHIMOTO, Tokyo (JP); Keisuke SUZUKI, Tokyo (JP)

(73) Assignee: NEC Corporation, Tokyo (JP)

(21) Appl. No.: 19/324,713

(22) Filed: Sep. 10, 2025

(30) Foreign Application Priority Data
Sep. 30, 2024 (JP) 2024-170665

(51) Int. Cl. G16H 50/70; G06N 7/01; G06Q 30/0283

(52) U.S. Cl. CPC G16H 50/70; G06N 7/01; G06Q 30/0283

(57) ABSTRACT

A path estimation device includes an acquisition unit, a generation unit, a path estimation unit, and an output unit. The acquisition unit acquires health-related data of a plurality of persons. The generation unit generates an occurrence probability distribution of the health-related data, based on the health-related data at a plurality of time points of each of the plurality of persons. The path estimation unit estimates a path between pieces of health-related data of a target person at different time points, on the generated occurrence probability distribution. The output unit outputs information regarding the estimated path. By including such a configuration, the path estimation device is capable of assisting decision making based on an estimation result of transition of the health-related data.

This application is based upon and claims the benefit of priority from Japanese Patent Application No. 2024-170665, filed on Sep. 30, 2024, the disclosure of which is incorporated herein in its entirety by reference.

### TECHNICAL FIELD

The present disclosure relates to a path estimation device or the like.

### BACKGROUND ART

To improve a health state or to prepare for future life, a future health state may be estimated. A health improvement path search device in WO 2022/085785 A1 estimates a health index based on a measurement value measured in a medical examination or the like. Then, the health improvement path search device in WO 2022/085785 A1 specifies a path from a current health index to an improved health index, based on a probability distribution of the estimated health index.

### SUMMARY

A path estimation device according to an aspect of the present disclosure includes an acquisition unit that acquires health-related data of a plurality of persons, a generation unit that generates an occurrence probability distribution of health-related data, based on the health-related data at a plurality of time points of each of the plurality of persons, a path estimation unit that estimates a path between pieces of health-related data of a target person at different time points, on the generated occurrence probability distribution, and an output unit that outputs information regarding the estimated path.

A path estimation method according to an aspect of the present disclosure includes acquiring health-related data of a plurality of persons, generating an occurrence probability distribution of health-related data, based on the health-related data at a plurality of time points of each of the plurality of persons, estimating a path between pieces of health-related data of a target person at different time points, on the generated occurrence probability distribution, and outputting information regarding the estimated path.

A non-transitory recording medium according to an aspect of the present disclosure records a path estimation program for causing a computer to execute processing for acquiring health-related data of a plurality of persons, processing for generating an occurrence probability distribution of health-related data, based on the health-related data at a plurality of time points of each of the plurality of persons, processing for estimating a path between pieces of health-related data of a target person at different time points, on the generated occurrence probability distribution, and processing for outputting information regarding the estimated path.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of a path estimation system according to the present disclosure;

FIG. 2 is a diagram illustrating an example of a configuration of a path estimation device according to the present disclosure;

FIG. 3 is a diagram schematically illustrating an example of processing for generating an occurrence probability distribution according to the present disclosure;

FIG. 4 is a diagram schematically illustrating an example of processing for estimating a path on the occurrence probability distribution according to the present disclosure;

FIG. 5 is a diagram schematically illustrating an example of the processing for estimating the path on the occurrence probability distribution according to the present disclosure;

FIG. 6 is a diagram illustrating an example of a path estimation result according to the present disclosure;

FIG. 7 is a diagram illustrating an example of the path estimation result according to the present disclosure;

FIG. 8 is a diagram illustrating an example of the path estimation result according to the present disclosure;

FIG. 9 is a diagram illustrating an example of the path estimation result according to the present disclosure;

FIG. 10 is a diagram illustrating an example of the path estimation result according to the present disclosure;

FIG. 11 is a diagram illustrating an example of the path estimation result according to the present disclosure;

FIG. 12 is a diagram illustrating an example of the path estimation result according to the present disclosure;

FIG. 13 is a diagram illustrating an example of the path estimation result according to the present disclosure;

FIG. 14 is a diagram illustrating an example of a display screen of the path estimation result according to the present disclosure;

FIG. 15 is a diagram illustrating an example of the display screen of the path estimation result according to the present disclosure;

FIG. 16 is a diagram illustrating an example of an operation flow of the path estimation device according to the present disclosure; and

FIG. 17 is a diagram illustrating an example of a hardware configuration of the path estimation device according to the present disclosure.

### EXAMPLE EMBODIMENT

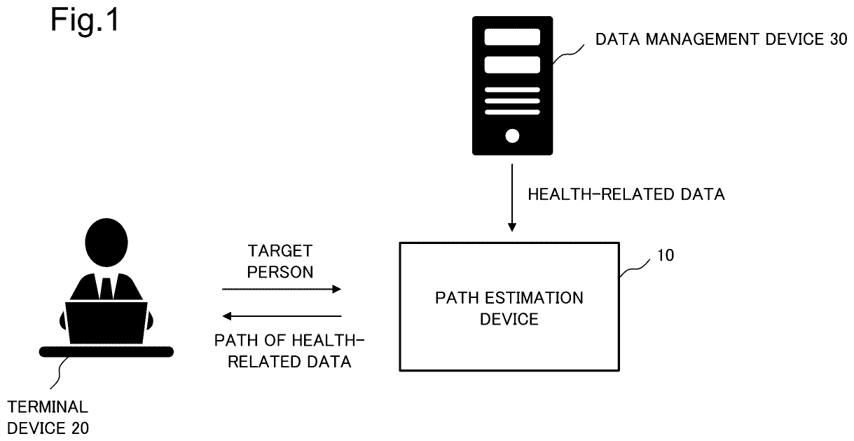

Example embodiments of the present disclosure will be described in detail with reference to the drawings. FIG. 1 is a diagram illustrating an example of a configuration of a path estimation system. The path estimation system includes a path estimation device 10, a terminal device 20, and a data management device 30. The path estimation device 10 is connected to the terminal device 20, for example, via a network. The path estimation device 10 is connected to the data management device 30, for example, via the network. A plurality of terminal devices 20 and a plurality of data management devices 30 may be provided. The number of terminal devices 20 and the number of data management devices 30 may be appropriately set.

The path estimation system estimates, for example, a path between pieces of health-related data of a target person at different time points. For example, the path estimation system estimates a path between pieces of health-related data of the target person at a current time point and a future time point. The path is, for example, transition of a value of the health-related data between the pieces of data.

The path estimation system estimates, for example, the path between the pieces of health-related data of the target person, on an occurrence probability distribution of the health-related data. The occurrence probability distribution of the health-related data is generated based on two or more items of health-related data, for example. The occurrence probability distribution of the health-related data is generated based on time-series data regarding health of a plurality of persons, for example. The health-related data is, for example, data indicating a health state of the target person. For example, the health-related data is a measurement value of a physical condition. The health-related data may be an index calculated from the measurement value of the physical condition.

The path between the pieces of health-related data of the target person is, for example, transition of data between the pieces of health-related data of the target person plotted on the occurrence probability distribution of the health-related data. That is, the path estimation system estimates how the health-related data of the target person transitions on the occurrence probability distribution of the health-related data. For example, the path estimation system may estimate a path of health-related data between adjacent time points, for pieces of data at a plurality of time points in chronological order.

The path between the pieces of health-related data of the target person is, for example, transition of pieces of data at time intervals finer than an interval between time points at which the data exists. For example, in a case where the data is a measurement value or an estimated value at a one-year interval, the path between the pieces of health-related data of the target person is transition of data for each month. For example, in a case where the occurrence probability distribution is divided into grids, health-related data at a time point t₁ exists in a grid P₁, and health-related data at a time point t₂ also exists in a grid P₂, the path between the pieces of health-related data of the target person is in order of grids through which the data passes through between P₁ and P₂ over time. For example, the target person is a person to be a target whose path between pieces of health-related data at different time points is estimated. The grid indicates, for example, one section in a case where each variable used for the occurrence probability distribution is divided for each predetermined value on the occurrence probability distribution. The predetermined value is, for example, set for each variable used for the occurrence probability distribution. The predetermined value is set so that it can be regarded that there is a meaningful difference in a case where a section including the data is changed, for each variable used for the occurrence probability distribution, for example. In this way, by estimating the path between the pieces of health-related data of the target person, the path estimation system can improve, for example, estimation accuracy of the transition of the data. In this case, the accuracy is, for example, a degree of precision of transition in a case where a state where the health-related data changes is expressed as the transition. For example, it is assumed that the accuracy be higher as a state where the health-related data continuously changes is represented as the data transition and the accuracy be lower in a case where a state where the health-related data discontinuously changes is represented as the data transition. For example, since the state where the health-related data continuously changes can be precisely represented by estimating the path between the pieces of health-related data, the path estimation system can improve the estimation accuracy of the transition of the data, for example.

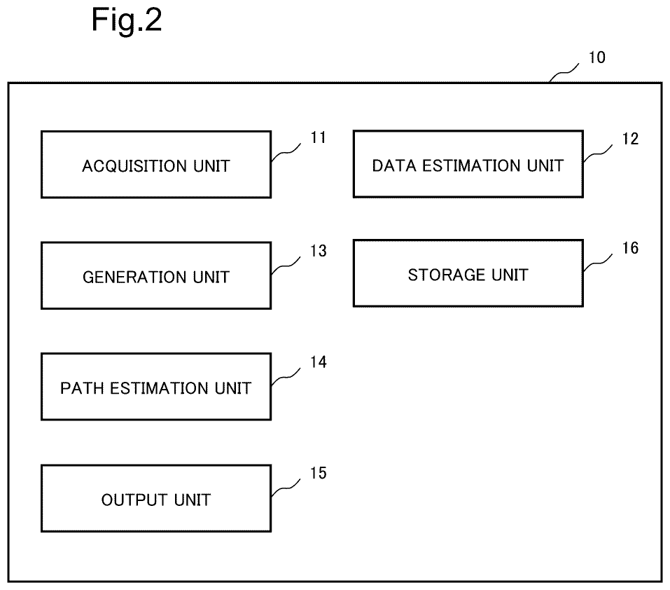

Here, a specific example of a configuration of the path estimation device 10 will be described. FIG. 2 illustrates an example of the configuration of the path estimation device 10. The path estimation device 10 includes an acquisition unit 11, a generation unit 13, a path estimation unit 14, and an output unit 15, as a basic configuration. The path estimation device 10 may further include, for example, a data estimation unit 12 and a storage unit 16.

The acquisition unit 11 acquires health-related data of a plurality of persons. For example, the acquisition unit 11 acquires the health-related data in a state in which information indicating an attribute of the person related to each piece of the health-related data is associated. The attribute is, for example, information indicating a group in which a difference in tendency of health-related data may occur due to a difference in the attribute. For example, tendency of health-related data may be different between a person who lives in a cold region and a person who lives in a warm region. In such a case, for example, information indicating a place of residence is used as the attribute. The attribute is, for example, information of one or more items of age, gender, place of residence, nationality, occupation, previous disease, and family history of previous diseases. The attribute is not limited to the above. For example, the acquisition unit 11 acquires the health-related data for each of the plurality of persons, from the data management device 30.

The health-related data is, for example, data indicating a physical condition. The health-related data may include the index calculated from the measurement value. The health-related data is, for example, data of one or more items among a result of a medical examination, a result of a medical interview, a test value in a hospital, vital data, presence or absence of onset of a disease, a probability of onset of a disease, opinions of a physician, a motor function, a degree of progression of dementia, a degree of progression of frailty, necessity of care, and a degree of necessary care. The medical examination result is, for example, data of one or a plurality of items among a height, a weight, a visual acuity, a blood pressure, an abdominal circumference, a hearing acuity, a measurement value in a blood test, an image diagnosis result, and a physician interview result measured in a medical examination. The degree of progression of dementia is, for example, an index indicating a degree of progression of dementia. For example, as the degree of progression of dementia increases, a risk of causing a trouble in daily life due to dementia increases. The degree of progression of frailty is, for example, an index indicating a degree of progression of frailty. For example, as the degree of progression of frailty increases, a risk of causing a trouble in daily life due to frailty increases.

The health-related data may include a cost necessary for maintaining a health state or a necessary cost according to the health state. The cost necessary for maintaining the health state or the necessary cost according to the health state is, for example, a medical care cost or living cost. For example, as the degree of progression of dementia increases, the medical care cost or the living cost may increase. As the degree of progression of frailty increases, the medical care cost or the living cost may increase. The cost necessary for maintaining the health state or the necessary cost according to the health state is not limited to the above. In addition, the health-related data is not limited to the above.

The acquisition unit 11 may acquire time-series data of the health-related data of each of the plurality of persons. For example, in a case where the health-related data is the result of the medical examination and the medical examination is conducted once a year, the acquisition unit 11 acquires a result of the medical examination per fiscal year for each of the plurality of persons. For example, the acquisition unit 11 acquires data of transition of the results of the medical examinations when the age increases, for each of the plurality of persons, as the time-series data of the health-related data.

The acquisition unit 11 acquires, for example, the health-related data of the target person. The acquisition unit 11 acquires, for example, information for designating the target person, from the terminal device 20. Then, the acquisition unit 11 acquires health-related information of the designated target person, for example, from the data management device 30. The information for designating the target person may include an attribute of the target person. The acquisition unit 11 may acquire the health-related data of the target person, from the terminal device 20.

The acquisition unit 11 may acquire information for designating a target point. The target point is, for example, a grid to be an ending point of a path in a case where the path between the pieces of health-related data is estimated on the occurrence probability distribution. The acquisition unit 11 acquires, for example, the information for designating the target point, from the terminal device 20. The acquisition unit 11 may acquire information for designating a grid to be a starting point of the path in a case where the path between the pieces of health-related data is estimated on the occurrence probability distribution. The acquisition unit 11 acquires, for example, the information for designating the grid to be the starting point of the path, from the terminal device 20.

The acquisition unit 11 may acquire information for designating a grid to be masked. The masking is, for example, processing for removing the grid set as the masking target from an estimation target of the path between the pieces of health-related data. That is, the masking is, for example, processing for setting a grid that blocks passage of a path regarding the health. The acquisition unit 11 acquires, for example, the information for designating the grid to be masked, from the terminal device 20. For example, the acquisition unit 11 may acquire information for designating a grid to be excluded from the masking target, from among the grids to be masked. The acquisition unit 11 may acquire information for designating a variable to be masked, as the information for designating the masking target. The acquisition unit 11 acquires, for example, the information for designating the grid to be excluded from among the masking targets, from the terminal device 20.

For example, the data estimation unit 12 estimates the time-series data of the health-related data of each of the plurality of persons, based on the health-related data of each of the plurality of persons. The time-series data estimated from the health-related data of each of the plurality of persons is, for example, used to generate a probability density distribution of the health-related data by the generation unit 13. The time-series data estimated from the health-related data of each of the plurality of persons is, for example, data at a time point later in chronological order than the health-related data to be a basis of the estimation. That is, for example, the data estimation unit 12 estimates time-series data of the health-related data at a future time point than a time point when the health-related data is measured, for the health-related data of each of the plurality of persons.

For example, the data estimation unit 12 estimates the time-series data of the health-related data of each of the plurality of persons, using a data estimation model. The data estimation model is a machine learning model that estimates the time-series data of the health-related data from input health-related data.

For example, the data estimation unit 12 estimates the time-series data of the health-related data of the target person, based on the health-related data of the target person. For example, the data estimation unit 12 estimates the time-series data of the health-related data of the target person, using the data estimation model. The data estimation model that estimates the time-series data of the health-related data of the target person is, for example, the same as a data estimation model that estimates the time-series data of the health-related data of each of the plurality of persons. The data estimation model that estimates the time-series data of the health-related data of the target person may be, for example, a machine learning model different from the data estimation model that estimates the time-series data of the health-related data of each of the plurality of persons. The different machine learning model is, for example, a machine learning model of which at least one of learning data and a learning algorithm is different.

The data estimation model is, for example, a machine learning model that estimates the time-series data of the health-related data at a time point later than input data in chronological order, using the health-related data as the input data. The data estimation model may be a machine learning model that estimates the time-series data of the health-related data at the time point later than the input data in chronological order, using the time-series data of the health-related data as the input data. For example, in a case where the health-related data is the result of the medical examination, the data estimation unit 12 estimates a result of a medical examination per fiscal year when the medical examination is conducted, for 20 years from a next year, based on results of medical examinations for 5 years up to this year. The data estimation model may be generated for each attribute of a person to be an estimation target, for example. For example, the data estimation model is generated by deep learning using a neural network. A machine learning algorithm for generating the data estimation model is not limited to the above. The data estimation model is generated, for example, by a device outside the path estimation device 10. The data estimation model may be generated by learning means (not illustrated) in the path estimation device 10.

The data estimation unit 12 may estimate the time-series data of the health-related data using a function used to calculate the health-related data. For example, the data estimation unit 12 calculates the health-related data of at each time point of the estimation target in chronological order, using a function with an elapsed time from the current time point and the health-related data at the current time point as explanatory variables. Then, for example, the data estimation unit 12 estimates the time-series data of the health-related data, by generating the time-series data based on the calculated health-related data at each time point of the estimation target in chronological order. The function used to calculate the health-related data is, for example, generated by regression analysis using the time-series data of the health-related data. An algorithm used to generate the function used to calculate the health-related data is not limited to the above.

The generation unit 13 generates the occurrence probability distribution of the health-related data, based on the plurality of pieces of the health-related data at the plurality of time points of each of the plurality of persons. For example, the generation unit 13 generates the occurrence probability distribution of the health-related data, based on the time-series data of the health-related data of each of the plurality of persons estimated by the data estimation unit 12. For example, the generation unit 13 generates the occurrence probability distribution of the health-related data of the plurality of persons, based on the time-series data of the health-related data of each of the plurality of persons, estimated by the data estimation model. The generation unit 13 may generate the occurrence probability distribution of the health-related data, based on time-series data of health-related data of each of the plurality of persons, acquired as measurement data. The generation unit 13 may generate the occurrence probability distribution of the health-related data, based on the data estimated by the data estimation model and the measurement data.

For example, in a case where two items of data among the health-related data are used as variables in the occurrence probability distribution, the generation unit 13 generates the occurrence probability distribution, on a grid having each of the two items of data as an axis. For example, in a case where the health-related data is the degree of progression of dementia and the degree of progression of frailty, the generation unit 13 classifies the health-related data into grids obtained by dividing the indices. For example, in a case where the health-related data is an average blood pressure and HbA1c, the generation unit 13 classifies the health-related data into grids obtained by dividing data of the average blood pressure by each 5 mmHg and data of the HbA1c by each 0.1%. Then, the generation unit 13 generates the occurrence probability distribution, by calculating an occurrence probability for each grid. For example, the generation unit 13 calculates the occurrence probability for each grid, by dividing the number of pieces of data classified into each grid by the total number of pieces of data. The health-related data used to generate the occurrence probability distribution may include three or more items. An item used as the variable of the occurrence probability distribution, in the health-related data, is not limited to the above.

For example, the generation unit 13 generates the occurrence probability distribution of the health-related data, using the time-series data of the health-related data of each of the plurality of persons, as independent data regardless of the person and the time point in chronological order. That is, for example, the generation unit 13 generates the occurrence probability distribution of the health-related data, using the health-related data at each of the plurality of time points of each of the plurality of persons, as the independent data. For example, in a case where there are pieces of health-related data at n time points for each of M persons, the generation unit 13 generates the occurrence probability distribution of the health-related data, using M×n pieces of data.

For example, in a case where the health-related data of each of the plurality of persons is data for each fiscal year, the generation unit 13 generates the occurrence probability distribution of the health-related data using pieces of data of the same person in different fiscal years as pieces of data independent from each other. For example, in a case where health-related data of a person A for n years is A1, A2, . . . , and An and health-related data of a person B for n years is B1, B2, . . . , and Bn, the generation unit 13 generates the occurrence probability distribution of the health-related data, using A1, A2, . . . , An, B1, B2, . . . , and Bn. By generating the occurrence probability distribution in this way, the time-series transition of the health-related data may be reflected on the occurrence probability distribution.

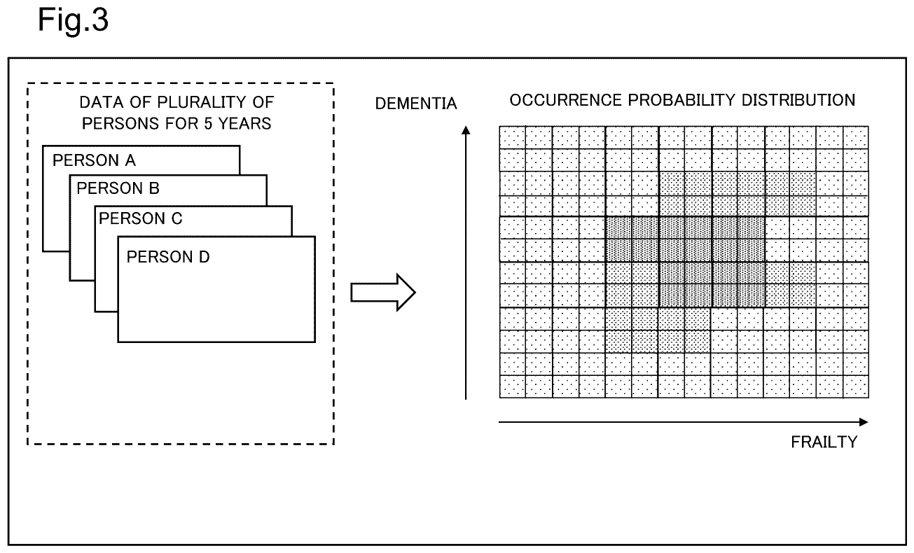

FIG. 3 is a diagram schematically illustrating an example of processing for generating the occurrence probability distribution. In the example in FIG. 3, the occurrence probability distribution is generated, based on the degree of progression of frailty and the degree of progression of dementia for each of the plurality of persons. In the example in FIG. 3, for example, the generation unit 13 generates the occurrence probability distribution with the degree of progression of frailty and the degree of progression of dementia as axes. In the example in FIG. 3, for example, the generation unit 13 generates the occurrence probability distribution using data of each of the plurality of persons for 5 years. In the example of the occurrence probability distribution in FIG. 3, the occurrence probability is higher in a grid with a darker color. For example, the generation unit 13 generates the occurrence probability distribution using data of each of the plurality of persons for 1 year as 1 piece of data.

For example, the generation unit 13 generates the occurrence probability distribution for each combination of the items of the health-related data. The combination of the items of health-related data used to generate the occurrence probability distribution is set, for example, based on a magnitude of an effect on daily life in a case where a symptom related to the item occurs. The combination of the items of the health-related data used to generate the occurrence probability distribution may be set, for example, based on an amount of a medical care cost in a case where a symptom related to the item occurs. The combination of the items of the health-related data used to generate the occurrence probability distribution may be set based on an occurrence rate of a disease related to the item.

The generation unit 13 may generate an occurrence probability distribution for each attribute. For example, the generation unit 13 generates an occurrence probability distribution for each gender. The generation unit 13 may generate the occurrence probability distribution for each age group to be a starting point of estimation. For example, in a case where a path of health-related data before a person in one's 50's becomes 70 years old is estimated, the generation unit 13 generates the occurrence probability distribution, using health-related data of the person in one's 50's and a predicted value of the health-related data each time when an age predicted from the health-related data increases. The generation unit 13 may generate an occurrence probability distribution for each residential area. The attribute used to generate the occurrence probability distribution is not limited to the above. For example, the generation unit 13 saves the generated occurrence probability distribution in the storage unit 16, in association with the attribute.

The path estimation unit 14 estimates the path between the pieces of health-related data of the target person at the different time points, on the generated occurrence probability distribution. For example, in a case where the health-related data is data for each year, the path estimation unit 14 estimates a path between the pieces of data for each year.

For example, the path estimation unit 14 extracts a pattern of a path between two points of which a path between the pieces of data is estimated. For example, the path estimation unit 14 extracts the pattern of the path so as not to pass through the same grid twice or more. The path estimation unit 14 may extract the pattern of the path so that the health-related data does not proceed to an improving direction. For example, in a case where the health-related data is the average blood pressure, the path estimation unit 14 estimates the pattern of the path so as to proceed to either one of a direction in which a numerical value increases or a direction in which the numerical value is the same. This is because the average blood pressure tends to increase as the age increases, for example. The path estimation unit 14 may extract a path of which the number of grids to be passed through is less than a predetermined number, as the pattern of the path. The predetermined number is set, for example, based on the number of grids existing between the two points of which the path is estimated. For example, in a case where two points for which a path is estimated are located at diagonal corners of a 3×3 grid, a possibility of passing through a path outside the 3×3 grid is low, and a possibility of passing through the same point in the grid a plurality of times is low. In such a case, the predetermined number is, for example, set as “4” that is the number of grids required to reach one point from another point as satisfying the above condition.

The path estimation unit 14 calculates, for example, the probability of passing through each extracted path pattern, as a passage probability. For example, the path estimation unit 14 calculates the passage probability by multiplying the occurrence probability of each grid on the path, for each path. Then, for example, the path estimation unit 14 estimates a path with the highest passage probability as the path between the two points to be estimated. The path estimation unit 14 may estimate the plurality of paths between the pieces of health-related data of the target person to the target point, on the occurrence probability distribution. For example, the path estimation unit 14 estimates a path of which the passage probability satisfies a criterion as a candidate of the path between the pieces of data. The criterion of the passage probability is set, for example, so that the number of extracted candidates is a number that allows to examine each extracted candidate, while a path that may be passed through is widely extracted.

For example, in a case where there are the pieces of data at the plurality of time points in chronological order, the path estimation unit 14 estimates a path between two consecutive points in chronological order.

For example, it is assumed that there be data for five years, data of a first year be d₁, data of a second year be d₂, data of a third year be d₃, data of a fourth year be d₄, and data of a fifth year be d₅. In this case, the path estimation unit 14 estimates, for example, paths between d₁ and d₂, between d₂ and d₃, between d₃ and d₄, and between d₄ and d₅. Then, for example, the path estimation unit 14 estimates an entire path obtained by connecting the paths between the two consecutive points in chronological order, as a path between d₁ and d₅. For example, the path estimation unit 14 may branch a path from a partial path from a certain path between two consecutive points in chronological order and estimate the path, as another new path, by connecting the path to another path in the vicinity of the partial path.

The path estimation unit 14 may estimate a path to each of the plurality of target points of the health-related data of the target person on the occurrence probability distribution. The target point is, for example, a grid related to an estimated value of the highest age, in a case where the health-related data is estimated for each age of the target person. The target point may be, for example, a grid related to an estimated value of the health-related data of an age designated by a user, in the estimation of the health-related data in a case where the age of the target person increases. The target point may be, for example, a grid related to a value of health-related data that is favorably reached, in a case where the age of the target person increases. The target point may be designated by the user. For example, the acquisition unit 11 acquires the information for designating the target point from the terminal device 20.

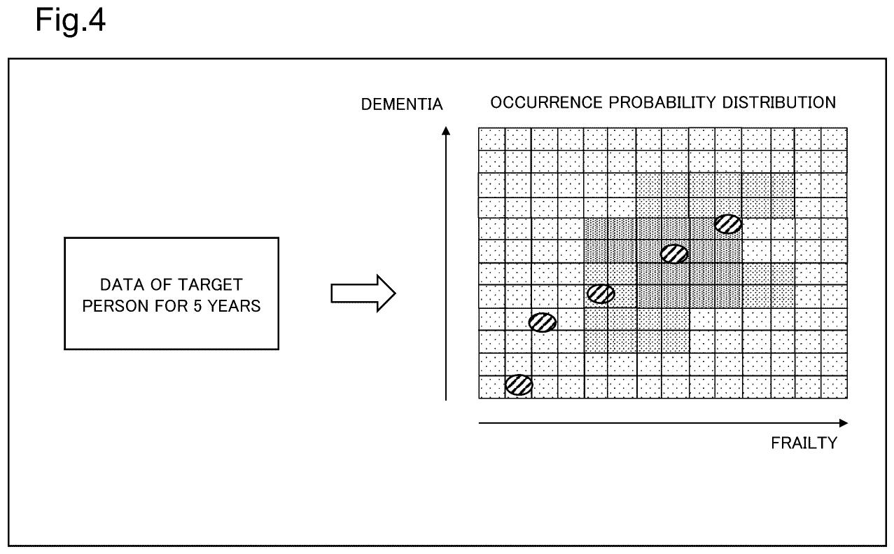

FIG. 4 is a diagram in which data of the target person per year is plotted on the occurrence probability distribution in the example in FIG. 3. In the example in FIG. 4, the occurrence probability distribution using each of the degree of progression of frailty and the degree of progression of dementia as the axis is generated, based on the degree of progression of frailty and the degree of progression of dementia for each of the plurality of persons. In the example in FIG. 4, the data of the target person for 5 years is plotted on the occurrence probability distribution, using each of the degree of progression of frailty and the degree of progression of dementia as the axis. In the example in FIG. 4, each piece of data of the target person is plotted on the occurrence probability distribution as a circle.

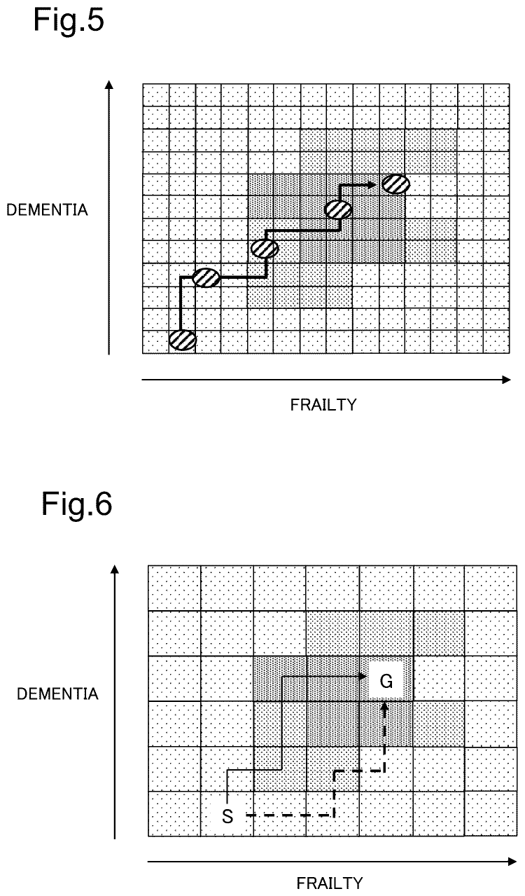

FIG. 5 is an example of the path estimation result between the pieces of data on the occurrence probability distribution in FIG. 4. In the example in FIG. 5, the occurrence probability distribution using each of the degree of progression of frailty and the degree of progression of dementia as the axis is generated, based on the degree of progression of frailty and the degree of progression of dementia for each of the plurality of persons. In the example in FIG. 5, the data of the target person for 5 years is plotted for each year on the occurrence probability distribution, using each of the degree of progression of frailty and the degree of progression of dementia as the axis. In the example in FIG. 5, a path estimation result between the pieces of data of the target person for each year plotted on the occurrence probability distribution is indicated by a solid line. In the example in FIG. 5, the path estimation result between the pieces of data of the target person for each year is illustrated on the occurrence probability distribution using each of the degree of progression of frailty and the degree of progression of dementia as the axis.

FIG. 6 is an example of an estimation result in a case where a plurality of paths is estimated, between the pieces of health-related data of the target person. In the example in FIG. 6, the occurrence probability distribution using each of the degree of progression of frailty and the degree of progression of dementia as the axis is generated, based on the degree of progression of frailty and the degree of progression of dementia for each of the plurality of persons. In the example in FIG. 6, estimation results of two paths from a starting point grid S to an ending point grid G are illustrated. In the example of the estimation result in FIG. 6, the estimation results of the two paths are indicated by a solid line and a broken line.

The path estimation unit 14 may estimate the plurality of paths, by masking at least one grid, among points on the occurrence probability distribution through which the estimated path passes. For example, the path estimation unit 14 estimates a new path, after masking at least one of the grids on the occurrence probability distribution through which the estimated path passes. Masking is, for example, to exclude a masked grid from path candidates. For example, the path estimation unit 14 sets a grid on the estimated path to be in a masked state. The masked grid is, for example, a grid excluded from an extraction target of the path candidate. For example, in a case where it is not possible to newly extract a pattern of a path, as a result of masking, the path estimation unit 14 ends the processing for estimating the path between the pieces of data.

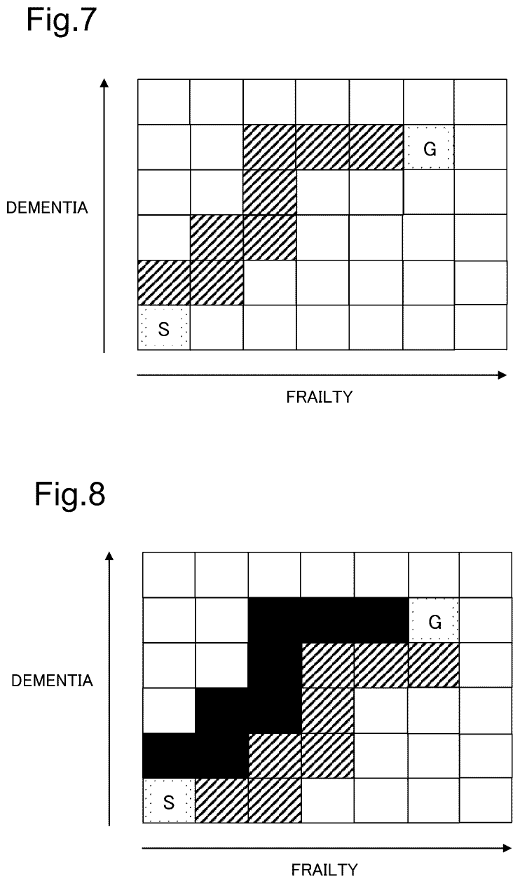

FIGS. 7 to 9 are examples of processing for masking a grid on a path estimated as the path between the pieces of data, in a case where the plurality of paths is estimated. In the examples in FIGS. 7 to 9, the occurrence probability distribution using each of the degree of progression of frailty and the degree of progression of dementia as the axis is generated, based on the degree of progression of frailty and the degree of progression of dementia for each of the plurality of persons. In the examples in FIGS. 7 to 9, the path estimation unit 14 estimates, for example, a path from the grid S to the grid G.

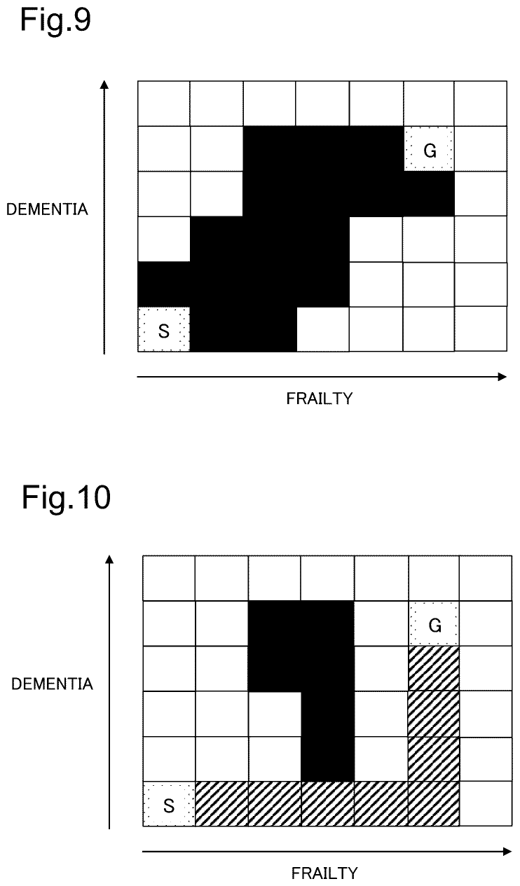

FIG. 7 is an example of an estimation result obtained by estimating a path with the highest passing probability, as a first path. In the example in FIG. 7, a hatched grid is the estimated grid on the path. FIG. 8 is an example of an estimation result obtained by estimating a path with the highest passing probability as a second path, in a case where grids through which the first path has passed are masked. In the example in FIG. 8, a black grid is a masked grid. In the example in FIG. 8, a hatched grid is a grid on the path estimated as the second path. FIG. 9 is an example of a state where the grids through which the first and second paths have passed are masked. In the example in FIG. 9, a black grid is a masked grid. In the example in FIG. 9, grids in both directions from the starting point grid S are the black grids. Therefore, in a state of the example in FIG. 9, there is no grid proceeded from the starting point grid S, it is not possible for the path estimation unit 14 to estimate third and subsequent paths. Therefore, in a case of the state as in the example in FIG. 9, the path estimation unit 14 does not estimate a new path, for example.

The path estimation unit 14 may exclude at least one of grids around the starting point or around the ending point, among the points on the occurrence probability distribution, from the masking target. FIG. 10 illustrates an example in which the grid around the starting point and the grid around the ending point are excluded from the masking target. In the example in FIG. 10, the occurrence probability distribution using each of the degree of progression of frailty and the degree of progression of dementia as the axis is generated, based on the degree of progression of frailty and the degree of progression of dementia for each of the plurality of persons. In the example in FIG. 10, the path estimation unit 14 estimates, for example, a path from the grid S to the grid G. In the example in FIG. 10, an example is illustrated in which the grid around the starting point and the grid around the ending point are excluded from the masking target and grids through which the first path and the second path have passed are masked. For example, in a state of the example in FIG. 9, since, around the grid at the ending point, there is only a path that goes around the masked grid, path estimation accuracy is lowered in a case where the third and subsequent paths are estimated. On the other hand, in the example in FIG. 10, since the grid around the starting point and the grid around the ending point are excluded from the masking target, more paths can be estimated than that in a case where the above grids are masked.

The path estimation unit 14 may estimate the path between the pieces of data, by masking at least one variable side point, among the points on the occurrence probability distribution. For example, the path estimation unit 14 estimates the path between the pieces of data, by masking at least one variable side point from a branching point of the path, among the points on the occurrence probability distribution. For example, in a case where the occurrence probability distribution has two variables, grids from a grid to be a starting point on one variable side to another variable side are masked. The masking direction is, for example, set to a side where it is not favorable for the health-related data to proceed. For example, in a case of estimating a path in a case where the progression of dementia is avoided, the path estimation unit 14 masks the variable side indicating the degree of progression of dementia. The variable to be masked is, for example, designated by the user.

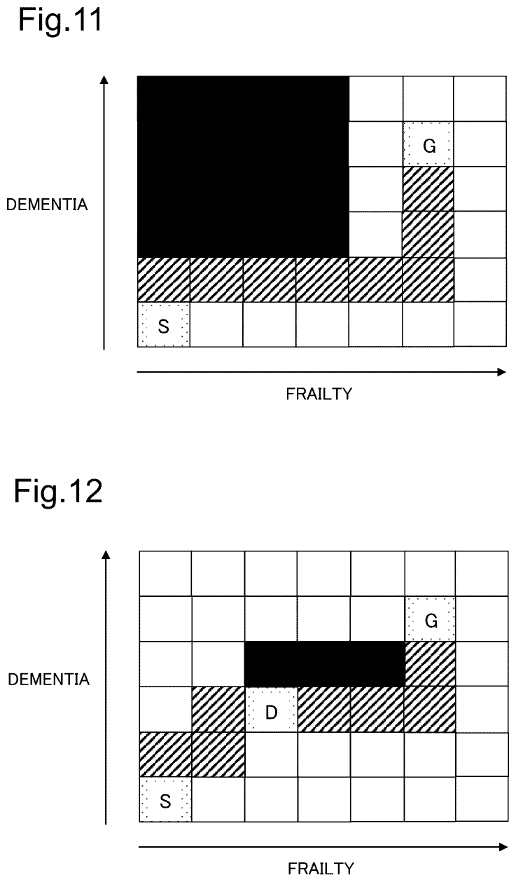

FIG. 11 is an example in which the path is estimated by masking a point on a side of one variable of two variables. In the example in FIG. 11, the occurrence probability distribution using each of the degree of progression of frailty and the degree of progression of dementia as the axis is generated, based on the degree of progression of frailty and the degree of progression of dementia for each of the plurality of persons. In the example in FIG. 11, the path estimation unit 14 estimates, for example, a path from the grid S to the grid G. In the example in FIG. 11, grids proceeding to a side where the degree of progression of dementia is higher, among the degree of progression of dementia and the degree of progression of frailty, are masked. Therefore, in the example in FIG. 11, the path estimation unit 14 estimates a path on a side where the degree of progression of frailty becomes higher. In this way, by performing masking in one direction, the path estimation unit 14 can estimate a path, for example, in a case of transition favorable for the target person. By performing masking in one direction, the path estimation unit 14 may estimate a path, in a case of transition that the target person should avoid.

FIG. 12 is an example in which the path between the pieces of data is estimated, by masking grids on a side of one variable of two variables from the branching point. In the example in FIG. 12, the occurrence probability distribution using each of the degree of progression of frailty and the degree of progression of dementia as the axis is generated, based on the degree of progression of frailty and the degree of progression of dementia for each of the plurality of persons. In the example in FIG. 12, the path estimation unit 14 estimates, for example, a path from the grid S to the grid G. In the example in FIG. 12, near a branching point D, a grid proceeding toward a side where the degree of progression of dementia increases is masked. Therefore, in the example in FIG. 12, the path estimation unit 14 estimates a path on a side where the degree of progression of frailty becomes higher. In this way, by performing masking in one direction near the branching point, for example, the path estimation unit 14 can estimate the path in a case of the transition favorable for the target person at and after the branching point. In addition, by performing masking in one direction near the branching point, for example, the path estimation unit 14 may estimate the path in a case of the transition that the target person should avoid at and after the branching point.

The path estimation unit 14 may estimate a path to the target point of the health-related data of the target person on the occurrence probability distribution, based on the occurrence probability distribution and the medical care cost estimated at each point on the occurrence probability distribution. For example, the path estimation unit 14 estimates a path of which an estimated value of the medical care cost is lower than that of other paths. For example, the path estimation unit 14 estimates the path of which the estimated value of the medical care cost is lower than that of the other paths, from among the paths of which the passage probability satisfies the criteria. A relationship between the health-related data used as the variable for the occurrence probability distribution and the medical care cost is, for example, set as data in a tabular format. For example, the path estimation unit 14 estimates a path of which an integrated value of the medical care cost on the path is the lowest.

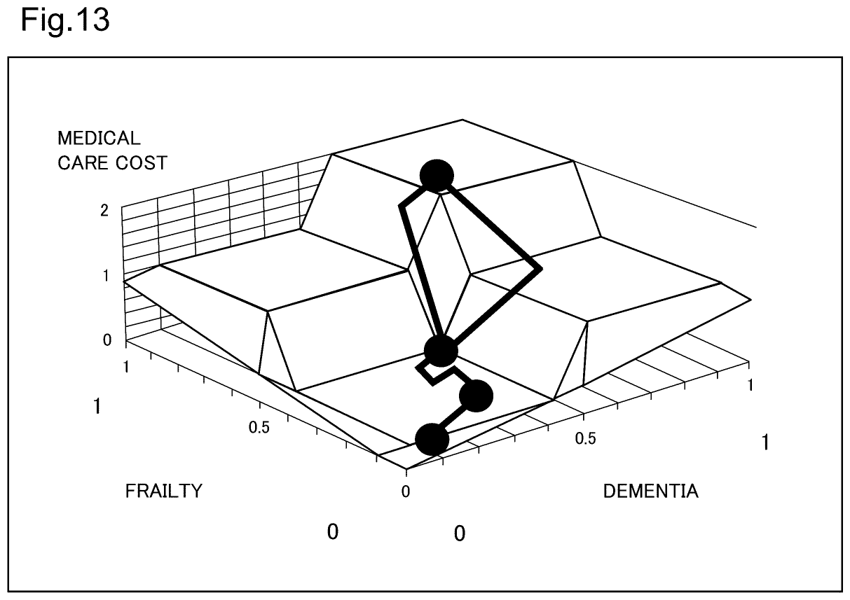

FIG. 13 is an example of the path estimated based on the occurrence probability distribution and the medical care cost estimated at each point on the occurrence probability distribution. In the example in FIG. 13, the path estimation result is illustrated on a map including the occurrence probability distribution using the degree of progression of dementia and the degree of progression of frailty as variables and the medical care cost. In FIG. 13, two paths are estimated at and after the branching point. In the example in FIG. 13, a speed of an increase in the medical care cost is different between a left path and a right path. Therefore, in the example in FIG. 13, the user can determine a favorable path, in consideration of the speed of the increase in the medical care cost.

The output unit 15 outputs information regarding the estimated path. For example, the output unit 15 superimposes the estimated path on the occurrence probability distribution and outputs them. The output unit 15 may output a candidate of the estimated path, based on a probability of passing through each path, as the information regarding the estimated path. For example, the output unit 15 outputs the candidate of the estimated path, in a display form different for each probability of passing through each path between the pieces of data. For example, the output unit 15 outputs the candidate of the estimated path, by changing at least one of the color and the line shape for each probability of passing through each path between the pieces of data. The output unit 15 may output the candidate of the estimated path by changing at least one of the color and the line shape for each stage of the probability of passing through each path between the pieces of data. How to output each path between the pieces of data is not limited to the above.

The output unit 15 may further superimpose and output a target path of the target person on the occurrence probability distribution, as the information regarding the estimated path. For example, the output unit 15 superimposes the path estimation result between the pieces of data and the target path of the target person on the occurrence probability distribution in a state of being distinguishable from each other and outputs them. For example, the output unit 15 outputs the path estimation result between the pieces of data and the target path of the target person, in a display form in which at least one of the color or the line shape is different. The display form of the path estimation result between the pieces of data and the target path of the target person is not limited to the above.

The output unit 15 outputs, for example, the information regarding the estimated path, to the terminal device 20. The output unit 15 may output the information regarding the estimated path, to a display device (not illustrated) connected to the path estimation device 10.

The output unit 15 may output a display screen for setting the target point. On the display screen for setting the target point, for example, in a case where the user performs a click operation on the occurrence probability distribution, a clicked grid is set as the target point. The output unit 15 may output a display screen for designating a grid to be masked. On the display screen for designating the grid to be masked, for example, in a case where the user performs the click operation on the occurrence probability distribution, the clicked grid is set as a masking target. For example, in a case where the user performs the click operation on the grid to be masked on the occurrence probability distribution, the clicked grid may be excluded from the masking target.

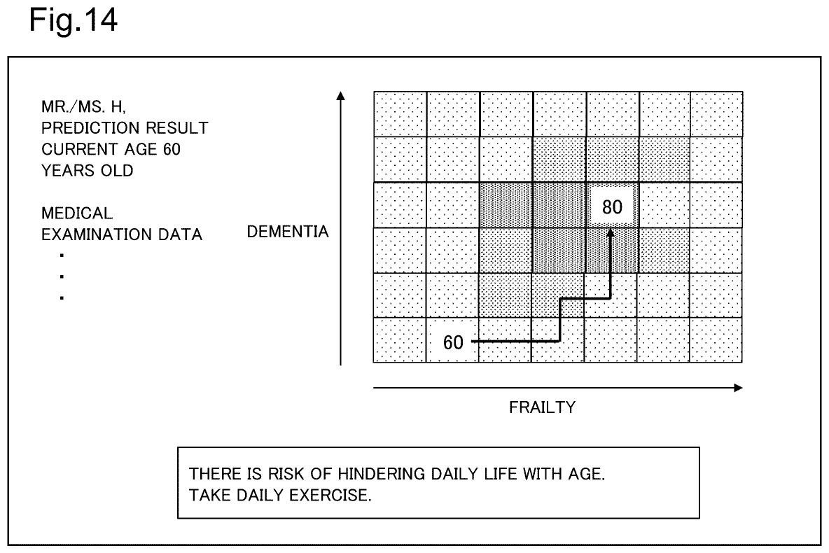

FIG. 14 is an example of a display screen of the path estimation result between the pieces of data. In the example of the display screen in FIG. 14, a name, a current age, and a latest medical examination result of the target person are displayed. In the example of the display screen in FIG. 14, on the occurrence probability distribution using the degree of progression of dementia and the degree of progression of frailty as the variables, a path estimation result until an age of a 60-year-old target person becomes 80 years old is displayed. In the example of the display screen in FIG. 14, advice regarding health of the target person is displayed in a lower portion of the display screen. For example, the output unit 15 outputs the display screen of the path estimation result as illustrated in the example in FIG. 14, to the terminal device 20. Then, the terminal device 20 outputs the path estimation result between the pieces of data, for example, to a display device (not illustrated). By outputting the display screen of the path estimation result as in the example of the display screen in FIG. 14, the path between the pieces of health-related data of the target person can be presented to the target person.

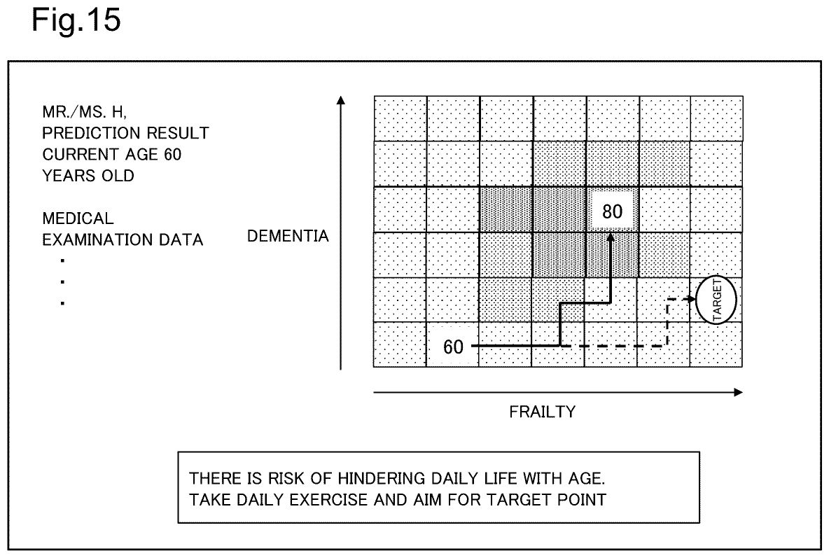

FIG. 15 is an example of the display screen that displays the path estimation result between the pieces of data and a target path. In the example of the display screen in FIG. 15, the name, the current age, and the latest medical examination result of the target person are displayed. The example of the display screen in FIG. 15 illustrates a path estimation result until the age of the 60-years-old target person becomes 80 years old. In the example of the display screen in FIG. 15, a path to a grid including a value to be a target when the target person becomes 80 years old is illustrated. For example, the output unit 15 outputs the display screen of the path estimation result as illustrated in the example in FIG. 15, to the terminal device 20. Then, the terminal device 20 outputs the path estimation result between the pieces of data, for example, to a display device (not illustrated). By outputting the display screen of the path estimation result as in the example of the display screen in FIG. 15, it is possible to present information indicating a difference between the path between the pieces of health-related data of the target person and a path used to reach a target health state, to the target person.

For example, the storage unit 16 saves data related to processing for estimating the path between the pieces of data at different time points on the occurrence probability distribution. For example, the storage unit 16 saves health-related data of a plurality of persons used to generate the occurrence probability distribution. For example, the storage unit 16 saves the time-series data of the health-related data of each of the plurality of persons. For example, the storage unit 16 saves the occurrence probability distribution generated by the generation unit 13. For example, the storage unit 16 saves the health-related data of the target person. The storage unit 16 saves, for example, the time-series data of the health-related data of the target person. For example, the storage unit 16 saves the path estimation result between the pieces of data at the different time points. For example, the storage unit 16 saves the data estimation model. The data estimation model may be saved in storage means outside the path estimation device 10.

For example, the terminal device 20 is used by a person who uses the path estimation result between the pieces of data at the different time points. For example, the terminal device 20 acquires information regarding the path estimation result, from the path estimation device 10. Then, for example, the terminal device 20 outputs the information regarding the path estimation result, to a display device (not illustrated). The terminal device 20 may acquire the information for designating the target person, input by an operation of the user. The information for designating the target person is, for example, information for identifying each target person or an attribute of the target person. The information for designating the target person is not limited to the above. In a case of acquiring the information for designating the target person, the terminal device 20 outputs the information for designating the target person, to the acquisition unit 11 of the path estimation device 10.

The terminal device 20 may acquire information for designating a grid to be the target point, input by the operation of the user. In a case of acquiring the information for designating the grid to be the target point, the terminal device 20 outputs the information for designating the grid to be the target point, to the acquisition unit 11 of the path estimation device 10. The terminal device 20 may acquire the information for designating the grid to be the starting point of the path, input by the operation of the user. In a case of acquiring the information for designating the grid to be the starting point of the path, the terminal device 20 outputs the information for designating the grid to be the starting point of the path, to the acquisition unit 11 of the path estimation device 10.

The terminal device 20 may acquire the information for designating the grid to be masked, input by the operation of the user. In a case of acquiring the information for designating the grid to be masked, the terminal device 20 outputs the information for designating the grid to be masked, to the acquisition unit 11 of the path estimation device 10. The terminal device 20 may acquire the information for designating the grid to be excluded from the masking target, input by the operation of the user. In a case of acquiring the information for designating the grid to be excluded from the masking target, the terminal device 20 outputs the information for designating the grid to be excluded from the masking target, to the acquisition unit 11 of the path estimation device 10.

The person who uses the path estimation result between the pieces of data is, for example, by the target person, a person who gives an advice regarding health to the target person, or a person who gives advice regarding an asset to the target person. The person who gives advice to the target person is, for example, a medical worker, an insurance person, a human resource person, a financial planner, or a person in charge of a financial institution. The medical worker is a physician, nurse, physical therapist, pharmacist, laboratory technician, or even a consultant. The medical worker is not limited to the above. The person who gives advice to the target person is not limited to the above. The person who uses the terminal device 20 can easily perform decision making regarding the health of the target person, for example, by referring to the path estimation result between the pieces of data at the different time points.

As the terminal device 20, for example, a personal computer, a tablet computer, a smartphone, or a smartwatch can be used. An information processing device used for the terminal device 20 is not limited to the above.

The data management device 30 stores, for example, health-related data. For example, the data management device 30 stores health-related data in association with a measurement date of the health-related data and the attribute of the person related to the health-related data. The data management device 30 outputs the health-related data to the acquisition unit 11 of the path estimation device 10, for example.

In a case where the health-related data is the result of the medical examination, the data management device 30 saves, for example, an execution date of the medical examination, an attribute of the person who has received the medical examination, and a result of the medical examination in association with each other. The attribute is, for example, information of one or more items of age, gender, place of residence, nationality, occupation, previous disease, and family history of previous diseases. The attribute is not limited to the above. The execution date of the medical examination may be information indicated by a month and a year, or a fiscal year in which the medical examination has been conducted. The data management device 30 may save the result of the medical examination, as a database classified based on at least one of the execution date of the medical examination and the attribute of the person who has received the medical examination.

For example, the data management device 30 may save the result of the medical examination as anonymized information or pseudonymized information. The anonymized information is, for example, information processed so that an individual cannot be identified even if the anonymized information is collated with other information. The pseudonymized information is, for example, information processed in such a way that an individual cannot be identified by itself but can be identified when the information is collated with other information.

The data management device 30 saves, for example, a result of a medical examination performed in a predetermined group. The predetermined group is, for example, a group that conducts a medical examination. The predetermined group is, for example, a municipality, a company, an association, a cooperative association, a school, or a health insurance association. The predetermined group is not limited to the above. The data management device 30 may save results of the medical examination of a plurality of groups.

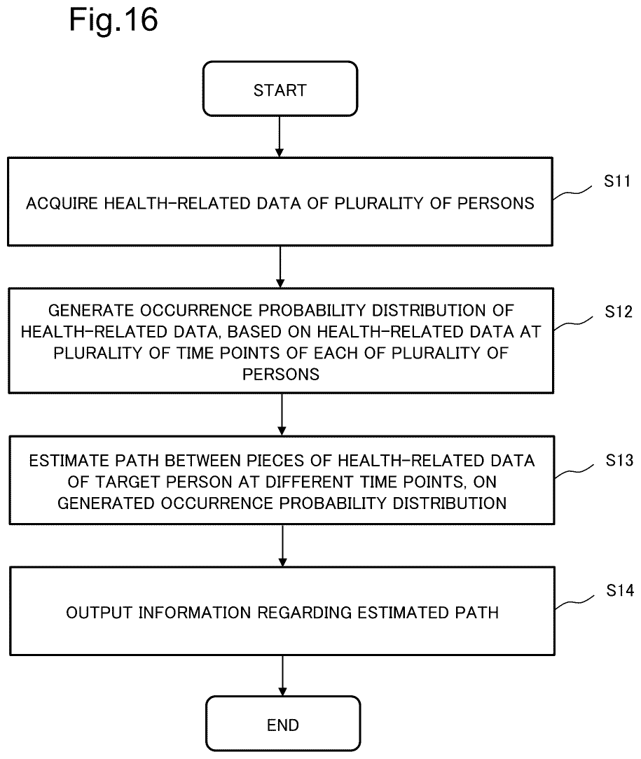

Processing for estimating the path between the pieces of data by the path estimation device 10 will be described. FIG. 16 is an example of an operation flow in the processing for estimating the path between the pieces of data by the path estimation device 10.

The acquisition unit 11 acquires the health-related data of the plurality of persons (step S11).

When the health-related data of the plurality of persons is acquired, the generation unit 13 generates the occurrence probability distribution of the health-related data, based on the health-related data at the plurality of time points of each of the plurality of persons (step S12). When the occurrence probability distribution of the health-related data is generated, the path estimation unit 14 estimates the path between the pieces of health-related data of the target person at the different time points, on the generated occurrence probability distribution (step S13).

When the path between the pieces of health-related data of the target person is estimated, the output unit 15 outputs the information regarding the estimated path (step S14). The output unit 15 outputs, for example, the information regarding the estimated path, to the terminal device 20.

Each processing of the path estimation device 10 may be executed in a distributed manner by a plurality of information processing devices connected via a network. For example, the processing of the data estimation unit 12 and the generation unit 13 and the processing of the path estimation unit 14 may be executed by different information processing devices. For example, the processing of the data estimation unit 12 and the processing of the generation unit 13 and the path estimation unit 14 may be executed by different information processing devices. Which information processing device performs each processing of the path estimation device 10 can be appropriately set.

The path estimation device 10 generates the occurrence probability distribution of the health-related data, based on the plurality of pieces of the health-related data at the plurality of time points of each of the plurality of persons. Then, the path estimation device 10 estimates the path between the pieces of health-related data of the target person at the different time points, on the generated occurrence probability distribution. In this way, the path estimation device 10 can improve the estimation accuracy of the transition of the health-related data, by estimating the path between the pieces of health-related data of the target person at the different time points, on the occurrence probability distribution generated based on the health-related data at the plurality of time points of each of the plurality of persons. That is, the path estimation device 10 can precisely represent the state where the health-related data changes, for example, by estimating the path between the pieces of health-related data.

By estimating the plurality of paths to the estimated value of the future health-related data of the target person, for example, the path estimation device 10 can present the path of the health-related data of the target person. By estimating the path of the health-related data of the target person to the target point, for example, the path estimation device 10 can output a path for causing the health state of the target person to be closer to the target point.

By estimating the path of the health-related data by excluding the estimated grids near the starting point and the ending point on the occurrence probability distribution from the masking target, for example, the path estimation device 10 can set more paths as estimation targets, than a case where the estimated grids near the starting point and the ending point are masked. The path of the health-related data can be estimated from a wide range of candidates. Therefore, for example, the path estimation device 10 can estimate the path of the health-related data from the wide range of candidates. Therefore, for example, the path estimation device 10 can improve the estimation accuracy of the path in a case where the path of the plurality of pieces of health-related data is estimated. That is, for example, the path estimation device 10 can precisely represent the state where the health-related data changes, in the path of the plurality of pieces of health-related data.

By estimating the path of the health-related data by masking an unfavorable side on the occurrence probability distribution, for example, the path estimation device 10 can output a path for causing the health-related data to proceed toward the favorable side. Therefore, for example, the path estimation device 10 can assist an action for improving health of the user who refers to the path estimation result of the health-related data. By including each configuration described above, the path estimation device 10 can assist decision making based on the estimation result of the transition of the health-related data.

By outputting the path of the health-related data and the medical care cost, the path estimation device 10 can output, for example, the estimation result of the health state in the future and the information regarding the medical care cost required for each estimated health state. Therefore, the path estimation device 10 can easily grasp the transition of the future health state and the required medical care cost. By estimating a path that minimizes the medical care cost, for example, the path estimation device 10 can output a path for causing the health state to proceed in a direction in which the medical care cost is minimized. Therefore, for example, the path estimation device 10 can assist decision making of the user who refers to the path estimation result.

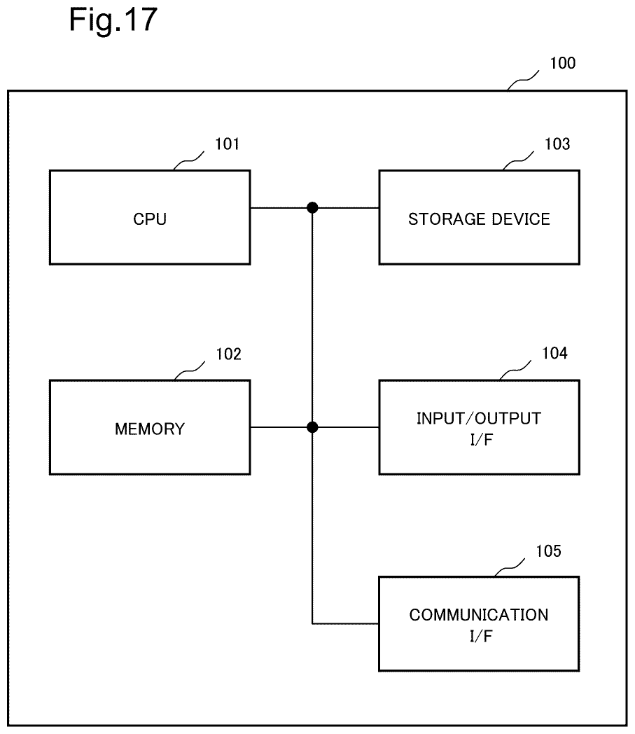

Each processing of the path estimation device 10 can be achieved by executing a computer program on a computer. FIG. 17 illustrates an example of a configuration of a computer 100 that executes a computer program for executing each processing of the path estimation device 10. The computer 100 includes a central processing unit (CPU) 101, a memory 102, a storage device 103, an input/output interface (I/F) 104, and a communication I/F 105.

The CPU 101 reads and executes the computer program for executing each processing from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores the computer program executed by the CPU 101 and data being processed. The storage device 103 stores the computer program executed by the CPU 101. The storage device 103 includes, for example, a non-volatile semiconductor storage device. As the storage device 103, another storage device such as a hard disk drive may be used. The input/output I/F 104 is an interface that receives an input from an operator and outputs a display screen or the like. The communication I/F 105 is an interface that transmits and receives data to and from the terminal device 20, the data management device 30, and other information processing devices. The terminal device 20 and the data management device 30 can also be configured as in the computer 100.

The computer program used for executing each processing can also be distributed by being stored in a computer-readable recording medium that non-transiently records data. The recording medium can include, for example, a magnetic tape for data recording or a magnetic disk such as a hard disk. The recording medium may include an optical disk such as a compact disc read only memory (CD-ROM). A non-volatile semiconductor storage device may be used as a recording medium.

To improve a health state or to prepare for future life, a future health state may be estimated. The future health state is estimated, for example, based on data indicating a current health state. In order to grasp the health state in more detail, transition of the health state from the present to the future may be estimated.

A health improvement path search device in WO 2022/085785 A1 estimates a health index based on a measurement value measured in a medical examination or the like. Then, the health improvement path search device in WO 2022/085785 A1 specifies a path from a current health index to an improved health index, based on a probability distribution of the estimated health index.

There is a case where it is difficult for the technique described in WO 2022/085785 A1 to improve estimation accuracy of transition of health-related data.

In order to solve the above problem, an object of the present disclosure is to provide a path estimation device or the like that can improve estimation accuracy of transition of health-related data.

According to the present disclosure, it is possible to improve estimation accuracy of transition of health-related data.

Some or all of the above example embodiments may be described as the following Supplementary Notes, but are not limited to the following.

### Supplementary Note 1

A path estimation device including:
an acquisition unit configured to acquire health-related data of a plurality of persons;
a generation unit configured to generate an occurrence probability distribution of health-related data, based on the health-related data at a plurality of time points of each of the plurality of persons;
a path estimation unit configured to estimate a path between pieces of health-related data of a target person at different time points, on the generated occurrence probability distribution; and
an output unit configured to output information regarding the estimated path.

### Supplementary Note 2

The path estimation device according to supplementary note 1, in which
the path estimation unit estimates a new path, by masking at least one grid, among points on the occurrence probability distribution through which the estimated path passes.

### Supplementary Note 3

The path estimation device according to supplementary note 2, in which
the path estimation unit estimates the path, by masking at least one variable side grid, among the points on the occurrence probability distribution.

### Supplementary Note 4

The path estimation device according to any one of supplementary notes 1 to 3, in which
the path estimation unit estimates a path to a target point of the health-related data of the target person on the occurrence probability distribution, based on the occurrence probability distribution and a medical care cost estimated in each grid on the occurrence probability distribution.

### Supplementary Note 5

The path estimation device according to any one of supplementary notes 1 to 4, in which
the path estimation unit estimates a plurality of paths to the target point of the health-related data of the target person on the occurrence probability distribution.

### Supplementary Note 6

The path estimation device according to any one of supplementary notes 1 to 5, in which
the path estimation unit estimates a path to each of a plurality of the target points of the health-related data of the target person on the occurrence probability distribution.

### Supplementary Note 7

The path estimation device according to any one of supplementary notes 1 to 6, in which
the output unit superimposes the estimated path on the occurrence probability distribution and outputs the estimated path and the occurrence probability distribution.

### Supplementary Note 8

The path estimation device according to any one of supplementary notes 1 to 7, further including:
a data estimation unit configured to estimate time-series data of the health-related data of each of the plurality of persons, using a machine learning model that estimates time-series data of health-related data at a later time point than input data in chronological order from input health-related data, in which
the generation unit generates an occurrence probability distribution of the health-related data of the plurality of persons, based on the time-series data of the health-related data of each of the plurality of persons.

### Supplementary Note 9

The path estimation device according to supplementary note 4, in which
the path estimation unit estimates a path of which an estimated value of the medical care cost is lower than other paths.

### Supplementary Note 10

The path estimation device according to any one of supplementary notes 1 to 9, in which
the output unit outputs a candidate of the estimated path, based on a probability of passing through each path.

### Supplementary Note 11

The path estimation device according to supplementary note 3, in which
the path estimation unit estimates the path, by masking at least one variable side point from a branching point of the path, among the points on the occurrence probability distribution.

### Supplementary Note 12

The path estimation device according to supplementary note 7, in which
the output unit superimposes a target path of the target person on the occurrence probability distribution and outputs the target path and the occurrence probability distribution.

### Supplementary Note 13

A path estimation method including: acquiring health-related data of a plurality of persons;
generating an occurrence probability distribution of health-related data, based on the health-related data at a plurality of time points of each of the plurality of persons;
estimating a path between pieces of health-related data of a target person at different time points, on the generated occurrence probability distribution; and
outputting information regarding the estimated path.

### Supplementary Note 14

A non-transitory recording medium for recording a path estimation program for causing a computer to execute:
processing for acquiring health-related data of a plurality of persons;
processing for generating an occurrence probability distribution of health-related data, based on the health-related data at a plurality of time points of each of the plurality of persons;
processing for estimating a path between pieces of health-related data of a target person at different time points, on the generated occurrence probability distribution; and
processing for outputting information regarding the estimated path.

Some or all of the configurations described in Supplementary Notes 2 to 12 dependent on the above-described Supplementary Note 1 can also be dependent on Supplementary Notes 13 and 14 by the same dependency relationship as in Supplementary Notes 2 to 12. Furthermore, some or all of the configurations described as the Supplementary Notes can be similarly dependent on not only the Supplementary Notes 1, 13, and 14, but also various pieces of hardware and software, and various recording means or systems for recording software without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A path estimation device comprising:
at least one memory storing instructions; and
at least one processor configured to access the at least one memory and execute the instructions to:
acquire health-related data of a plurality of persons;
generate an occurrence probability distribution of health-related data, based on the health-related data at a plurality of time points of each of the plurality of persons;
estimate a path between pieces of health-related data of a target person at different time points, on the generated occurrence probability distribution; and
output information regarding the estimated path.

2. The path estimation device according to claim 1, wherein
the at least one processor is further configured to execute the instructions to:
estimate a new path, by masking at least one grid, among grids on the occurrence probability distribution through which the estimated path passes.

3. The path estimation device according to claim 2, wherein
the at least one processor is further configured to execute the instructions to:
estimate the path, by masking at least one variable side grid, among points on the occurrence probability distribution.

4. The path estimation device according to claim 1, wherein
the at least one processor is further configured to execute the instructions to:
estimate a path to a target point of the health-related data of the target person on the occurrence probability distribution, based on the occurrence probability distribution and a medical care cost estimated in each grid on the occurrence probability distribution.

5. The path estimation device according to claim 1, wherein
the at least one processor is further configured to execute the instructions to:
estimate a plurality of paths to a target point of the health-related data of the target person on the occurrence probability distribution.

6. The path estimation device according to claim 1, wherein
the at least one processor is further configured to execute the instructions to:
estimate a path to each of a plurality of target points of the health-related data of the target person on the occurrence probability distribution.

7. The path estimation device according to claim 1, wherein
the at least one processor is further configured to execute the instructions to:
superimpose the estimated path on the occurrence probability distribution and outputs the estimated path and the occurrence probability distribution.

8. The path estimation device according to claim 1, further comprising:
the at least one processor is further configured to execute the instructions to:
estimate time-series data of the health-related data of each of the plurality of persons, using a machine learning model that estimates time-series data of health-related data at a later time point than input data in chronological order from input health-related data; and
generate an occurrence probability distribution of the health-related data of the plurality of persons, based on the time-series data of the health-related data of each of the plurality of persons.

9. The path estimation device according to claim 4, wherein
the at least one processor is further configured to execute the instructions to:
estimate a path of which an estimated value of the medical care cost is lower than other paths.

10. The path estimation device according to claim 1, wherein
the at least one processor is further configured to execute the instructions to:
output a candidate of the estimated path, based on a probability of passing through each path.

11. The path estimation device according to claim 3, wherein
the at least one processor is further configured to execute the instructions to:
estimate the path, by masking at least one variable side point from a branching point of the path, among the points on the occurrence probability distribution.

12. The path estimation device according to claim 7, wherein
the at least one processor is further configured to execute the instructions to:
superimpose a target path of the target person on the occurrence probability distribution and outputs the target path and the occurrence probability distribution.

13. A path estimation method comprising:
acquiring health-related data of a plurality of persons;
generating an occurrence probability distribution of health-related data, based on the health-related data at a plurality of time points of each of the plurality of persons;
estimating a path between pieces of health-related data of a target person at different time points, on the generated occurrence probability distribution; and
outputting information regarding the estimated path.

14. A non-transitory recording medium recording a path estimation program for causing a computer to execute:
processing for acquiring health-related data of a plurality of persons;
processing for generating an occurrence probability distribution of health-related data, based on the health-related data at a plurality of time points of each of the plurality of persons;
processing for estimating a path between pieces of health-related data of a target person at different time points, on the generated occurrence probability distribution; and
processing for outputting information regarding the estimated path.
